(19) United States
(12) Patent Application Publication
(10) Pub. No.: US 2024/0177851 A1
(43) Pub. Date: May 30, 2024

(54) INFORMATION ACQUISITION METHOD, INFORMATION PROVIDING METHOD, INFORMATION INTERACTION METHOD AND APPARATUS THEREOF

(71) Applicant: BOE TECHNOLOGY GROUP CO., LTD., Beijing (CN)

(72) Inventors: Yanyang Hu, Beijing (CN); Jiao Huang, Beijing (CN); Yubin Zhou, Beijing (CN)

(73) Assignee: BOE TECHNOLOGY GROUP CO., LTD., Beijing (CN)

(21) Appl. No.: 17/777,839

(22) PCT Filed: May 21, 2021
(86) PCT No.: PCT/CN2021/095322

§ 371 Date: May 18, 2022

(51) Int. Cl. G16H 40/67; G16H 50/30

(52) U.S. Cl. CPC G16H 40/67; G16H 50/30

(57) ABSTRACT

An information acquisition method, an information providing method, an information interaction method and apparatus thereof provided by the present disclosure, relate to the technical field of computer technology. The method includes: displaying a concerning user related to a login user, receiving a first input for the concerning user, and displaying health information of the concerning user in response to the first input.

### TECHNICAL FIELD

The present disclosure relates to the technical field of computer technology, and more particularly, to an information acquisition method, an information providing method, an information interaction method and an apparatus thereof.

### BACKGROUND

Currently, most of the technologies in the field of elderly care focus on transforming residential environment, and perform intelligent designs, which is suitable for elderly people of the residential places, it may provide a lot of convenience for elderly people by placing video monitoring, environment monitoring, physiological signal monitoring devices and facilities and so on in the residential environment. These user monitoring devices may alarm in time when there is fire, gas leak or such environmental emergency happens, and they may also monitor the physiological health situation of the elderly people in the residential environment, so that their families may know the living situation of the elderly people by the monitoring information.

### SUMMARY

The present disclosure provides an information acquisition method, an information providing method, an information interaction method and an apparatus thereof.

Some embodiments of the present disclosure provide an information acquisition method, the method includes:
displaying a concerning user related to a login user;
receiving a first input for the concerning user; and
displaying health information of the concerning user in response to the first input.

Optionally, the method further includes:
receiving a second input for a following functional identification;
displaying a user information input column in response to the second input;
receiving user information input into the user information input column; and
regarding a user indicated by the user information as the concerning user related to the login user.

Optionally, regarding the user indicated by the user information as the concerning user
related to the login user includes:
acquiring a verification result between the user indicated by the user information and the login user; and
when the verification result is that the verification is passed, regarding the user indicated by the user information as the concerning user related to the login user.

Optionally, before acquiring the verification result between the user indicated by the user information and the login user, the method further includes:
displaying a user registration column when the user indicated by the user information is not registered; and
receiving registration information input into the user registration column to register the user indicated by the user information.

Optionally, displaying the user information input column in response to the second input includes:
displaying functional description information in response to the second input; and
displaying the user information input column after receiving a confirmation input of the functional description information.

Optionally, the method further includes:
outputting prompt information of the health information when the health information of the concerning user satisfies a preset prompt condition.

Optionally, outputting the prompt information of the health information includes:
outputting the prompt information of the health information in a target prompt way, the target prompt way at least includes at least one of a short message prompt way, an email prompt way, a communication-application prompt way an audio prompt way, a video prompt way, a light prompt way and a vibration prompt way.

Optionally, when the preset prompt condition is a behavior-trajectory abnormality
condition, the health information includes: behavior-trajectory information of the concerning user, the prompt information includes: a behavior-trajectory abnormality prompt information;
outputting the prompt information of the health information when the health information of the concerning user satisfies the preset prompt condition includes:
outputting the behavior-trajectory abnormality prompt information of the concerning user when the behavior-trajectory information of the concerning user satisfies the behavior-trajectory abnormality condition.

Optionally, when the preset prompt condition is a health abnormality condition, the health information includes: physical indicators of the concerning user, the prompt information includes: health abnormality prompt information;
outputting the prompt information of the health information when the health information of the concerning user satisfies the preset prompt condition includes:
outputting the health abnormality prompt information of the concerning user when the physical indicators of the concerning user satisfy the health abnormality condition.

Optionally, when the preset prompt condition is a diagnosis and treatment abnormality condition, the health information includes: a drug taking record and a physical examination record of the concerning user, the prompt information includes: a diagnosis and treatment abnormality prompt information;
outputting the prompt information of the health information when the health information of the concerning user satisfies the preset prompt condition includes:
outputting the diagnosis and treatment abnormality prompt information of the concerning user when the drug taking record and the physical examination record of the concerning user satisfy the diagnosis and treatment abnormality condition.

Optionally, the method further includes:
displaying the health information of the login user.

An information providing method provided by some embodiments of the present disclosure, the method includes:
acquiring health information of a concerning user from user monitoring equipment;
determining a login user related to the concerning user; and
sending the health information to a client corresponding to the login user.

Optionally, the method further includes:
receiving user information sent by the client corresponding to the login user;
acquiring a trust value between a user indicated by the user information and the login user according to the user information; and
when the trust value is greater than a trust value threshold, determining a verification result between the user indicated by the user information and the login user is passed, and sending the verification result to the client.

Optionally, the user information at least includes: user identification information, user reply information and a user question quantity:
acquiring the trust value between the user indicated by the user information and the login user includes:
querying a user indicated by the user information according to the user identification information; and
acquiring the trust value between the user indicated by the user information and the login user according to the user reply information and the user question quantity.

Optionally, acquiring the trust value between the user indicated by the user information and the login user according to the user reply information and the user question quantity includes:
according to the user reply information and the user question quantity, acquiring the trust value between the user indicated by the user information and the login user through following formulas:

$$\begin{matrix}T = E ( Beta ( \begin{matrix}θ \vert r + 1 , & s + 1\end{matrix} ) ) = \frac{r + 1}{r + s + 2} \\ r = r 1 + r 2\end{matrix}$$

wherein, the T is the trust value between the user indicated by the user information and the login user, the r1 is a quantity of questions matched between the user reply information and standard question-answer information, the r2 is a sum of probability matched between the user reply information and the standard question-answer information, the s is a quantity of questions not matched between the user reply information and the standard question-answer information.

Optionally, after determining the verification result between the user indicated by the user information and the login user is passed, the method further includes:
establishing a following relationship among a target user account, the user indicated by the user information and an anonymous user, a user attribute of the anonymous user is the same as a user attribute of the user indicated by the user information.

Optionally, after acquiring the health information of the concerning user from the user monitoring equipment, the method further includes:
acquiring an abnormality level of the health abnormality condition when the health information satisfies an abnormal alarm condition; and
outputting alarm information according to an alarm way corresponding to the abnormality level.

Optionally, outputting the alarm information according to the alarm way corresponding to the abnormality level includes:
sending the alarm information to the login user when the abnormality level is a first abnormality level;
sending the alarm information to a community staff and the login user when the abnormality level is a second abnormality level; and
sending the alarm information to the community staff, first-aid personnel, and the login user when the abnormality level is a third abnormality level.

Some embodiments of the present disclosure provide an information interaction method, the method further includes:
a server acquires health information of a concerning user from user monitoring equipment;
the sever sends the health information to a client of a login user, and the login user is related to the concerning user;
the client displays the concerning user;
the client receives a target input for the concerning user; and
the client displays the health information of the concerning user in response to the target input.

Some embodiments of the present disclosure provide an information acquisition apparatus, the apparatus includes:
a homepage displaying module configured for displaying a concerning user related to a login user;
a receiving module configured for receiving a first input for the concerning user; and
an information displaying module configured for displaying health information of the concerning user in response to the first input.

Optionally, the apparatus further includes:
an association module is configured for:
receiving a second input for a following functional identification;
displaying a user information input column in response to the second input;
receiving user information input into the user information input column
regarding a user indicated by the user information as the concerning user related to the login user.

Optionally, the association module is further configured for:
acquiring a verification result between the user indicated by the user information and the login user;
when the verification result is that the verification is passed, regarding the user indicated by the user information as the concerning user related to the login user.

Optionally, the association module is further configured for:
displaying a user registration column when the user indicated by the user information is not registered;
receiving registration information input into the user registration column to register the user indicated by the user information.

Optionally, the association module is further configured for:
displaying functional description information in response to the second input;
displaying a user information input column after receiving a confirmation input for the functional description information.

Optionally, the information displaying module is further configured for:
outputting prompt information of the health information when the health information of the concerning user satisfies a preset prompt condition.

Optionally, the information displaying module is further configured for:
outputting the prompt information of the health information by a target prompt way, the target prompt way at least includes one of the followings: a short message prompt way, an email prompt way, a communication application prompt way, an audio prompt way, a video prompt way, a lighting prompt way, or a vibration way.

Optionally, when the preset prompt condition is a behavior-trajectory abnormality condition, the health information includes: behavior-trajectory information of the concerning user, the prompt information includes: behavior-trajectory abnormality prompt information:

The information displaying module is further configured for:
outputting the behavior-trajectory abnormality prompt information of the concerning user when the behavior-trajectory information of the concerning user satisfies the behavior-trajectory abnormality condition.

Optionally, when the preset prompt condition is a health abnormality condition, the health information includes: the physical indicators of the concerning user, the prompt information includes: health abnormality prompt information:

The information displaying module is further configured for:
outputting the health abnormality prompt information of the concerning user when the physical indicators of the concerning user satisfy the health abnormality condition.

Optionally, when the preset prompt condition is a diagnosis and treatment abnormality condition, the health information includes: a medication taking record and a physical examination record of the concerning user, the prompt information includes: diagnosis and treatment abnormality prompt information:

The information displaying module is further configured for:
outputting the diagnosis and treatment abnormality prompt information of the concerning user when the medication taking record and the physical examination record of the concerning user satisfy the diagnosis and treatment abnormality condition.

Optionally, the homepage displaying module is further configured for:
displaying the health information of the login user.

An information providing apparatus provided by an embodiment of the present disclosure, and the apparatus includes:
an acquisition module configured for acquiring health information of a concerning user from a user monitoring equipment;
a determining module configured for determining a login user related to the concerning user;
a sending module configured for sending the health information to a client corresponding to the login user.

Optionally, the apparatus further includes:
a verification module is configured for:
receiving user information sent from the client corresponding to the login user;
acquiring a trust value between a user indicated by the user information and the login user according to the user information;
when the trust value is greater than a trust value threshold, determining a verification result between the user indicated by the user information and the login user is passed, and sending the verification result to the client.

Optionally, the user information at least includes: user identification information, user reply information and the user question quantity:

The verification module is further configured for:
querying a user indicated by the user information according to the user identification information;
acquiring the trust value between the user indicated by the user information and the login user according to the user reply information and the user question quantity.

Optionally, the verification module is further configured for:
according to the user reply information and the user question quantity, acquiring the trust value between the user indicated by the user information and the login user through following formulas:

$$\begin{matrix}T = E ( Beta ( \begin{matrix}θ \vert r + 1 , & s + 1\end{matrix} ) ) = \frac{r + 1}{r + s + 2} \\ r = r 1 + r 2\end{matrix}$$

wherein, the T is the trust value between the user indicated by the user information and the login user, the r1 is a quantity of questions matched between the user reply information and standard question-answer information, the r2 is a sum of probability matched between the user reply information and the standard question-answer information, the s is a quantity of questions not matched between the user reply information and the standard question-answer information.

Optionally, the verification module is further configured for:
establishing a following relationship between a target user account, the user indicated by the user information and an anonymous user, a user attribute of the anonymous user is the same as the user indicated by the user information.

Optionally, the apparatus further includes:
an alarm module is configured for:
acquiring an abnormality level of the health abnormality condition when the health information satisfies an abnormal alarm condition;
outputting alarm information according to an alarm way corresponding to the abnormality level.

Optionally, the alarm module is further configured for:
sending the alarm information to the login user when the abnormality level is a first abnormality level;
sending the alarm information to a community staff and the login user when the abnormality level is a second abnormality level;
sending the alarm information to the community staff, first-aid personnel, and the login user when the abnormality level is a third abnormality level.

An information interaction system provided by an embodiment of the present disclosure, the system includes: a client a client, a server and monitoring equipment:

The server is configured for:
acquiring health information of a concerning user from the user monitoring equipment;
sending the health information to a client of a login user, the login user is related to the concerning user;

The client is configured for:
displaying the concerning user;
receiving a target input for the concerning user;
displaying the health information of the concerning user in response to the target input.

A computing and processing device provided by some embodiments of the present disclosure, wherein the device includes:
a memory in which a computer-readable code is stored; and
one or more processors, wherein when the computer-readable code is executed by the one or more processors, the computing and processing device executes the information acquisition method or the information providing method or the information interaction method as mentioned above.

A computer program provided by some embodiments of the present disclosure, wherein the computer program includes a computer-readable code that, when executed on a computing and processing device, causes the computing and processing device to execute the information acquisition method or the information providing method or the information interaction method as mentioned above.

A computer-readable medium provided by some embodiments of the present disclosure, wherein the computer-readable medium stores the information acquisition method or the information providing method or the information interaction method as mentioned above.

The above description is merely a summary of the technical solutions of the present disclosure. In order to more clearly know the elements of the present disclosure to enable the implementation according to the contents of the description, and in order to make the above and other purposes, features, and advantages of the present disclosure more apparent and understandable, the particular embodiments of the present disclosure are provided below.

### BRIEF DESCRIPTION OF THE DRAWINGS

In order to more clearly illustrate the technical solutions of the embodiments of the present disclosure or the prior art, the figures that are required to describe the embodiments or the prior art will be briefly introduced below. Apparently, the figures that are described below are embodiments of the present disclosure, and a person skilled in the art can obtain other figures according to these figures without paying creative work.

FIG. 1 schematically shows a first flow chart of an information acquisition method provided by some embodiments of the present disclosure:

FIG. 2 schematically shows a second flow chart of the information acquisition method provided by some embodiments of the present disclosure:

FIG. 3 schematically shows a first schematic diagram showing the effect of the information acquisition method provided by some embodiments of the present disclosure:

FIG. 4 schematically shows a second schematic diagram showing the effect of the information acquisition method provided by some embodiments of the present disclosure:

FIG. 5 schematically shows a third schematic diagram showing the effect of the information acquisition method provided by some embodiments of the present disclosure:

FIG. 6 schematically shows a fourth schematic diagram showing the effect of the information acquisition method provided by some embodiments of the present disclosure:

FIG. 7 schematically shows a third flow chart of the information acquisition method provided by some embodiments of the present disclosure:

FIG. 8 schematically shows a fifth schematic diagram showing the effect of the information acquisition method provided by some embodiments of the present disclosure:

FIG. 9 schematically shows a fourth flow chart of the information acquisition method provided by some embodiments of the present disclosure:

FIG. 10 schematically shows a sixth schematic diagram showing the effect of the information acquisition method provided by some embodiments of the present disclosure:

FIG. 11 schematically shows a first flow chart of an information providing method provided by some embodiments of the present disclosure:

FIG. 12 schematically shows a schematic diagram showing the principle of the information providing method provided by some embodiments of the present disclosure:

FIG. 13 schematically shows a second flow chart of the information providing method provided by some embodiments of the present disclosure:

FIG. 14 schematically shows a third flow chart of the information providing method provided by some embodiments of the present disclosure:

FIG. 15 schematically shows a fourth flow chart of the information providing method provided by some embodiments of the present disclosure:

FIG. 16 schematically shows a fifth flow chart of the information providing method provided by some embodiments of the present disclosure:

FIG. 17 schematically shows a flow chart of an information interaction method provided by some embodiments of the present disclosure:

FIG. 18 schematically shows a structural schematic diagram of an information acquisition apparatus provided by some embodiments of the present disclosure:

FIG. 19 schematically shows a structural schematic diagram of an information providing apparatus provided by some embodiments of the present disclosure:

FIG. 20 schematically shows a structural schematic diagram of an information interaction system provided by some embodiments of the present disclosure:

FIG. 21 schematically shows a block diagram of a computing and processing device for executing the methods provided by some embodiments of the present disclosure; and

FIG. 22 schematically shows a storage unit for holding or carrying a program code that executes methods provided by some embodiments of the present disclosure.

### DETAILED DESCRIPTION OF THE EMBODIMENTS

In order to make the above objects, features, and advantages of the present disclosure more obvious and understandable, the present disclosure will be described in further detail below with reference to the accompanying drawings and specific implementation ways. Obviously, the described embodiments are some, but not all, embodiments of the present disclosure. Based on the embodiments in the present disclosure, all other embodiments obtained by a person of ordinary skills in the art without inventive efforts fall within the scope of the present disclosure.

With the continuous delay of retirement age, people with working ability have longer working hours, so they spend less and less time to take care of elderly at home. In addition, the elderly people are unskilled to use intelligent equipment, which are still unable to meet the requirements of physical health and mental health of the elderly people living alone and the empty nest elderly people in the related art, such as the compliance of health monitoring/medication for the elderly with chronic diseases, the failure of finding the elderly in time when they are out of home for a long time, the failure of family members to watch the house surveillance video for a long time to discover the abnormal situation of the elderly at home, and the lack of communication between the elderly and their children leading to the mental health problems of the elderly. Moreover, the security of data grabs more attentions in recent years, a certain prevention for data leakage or malicious attacks should be done.

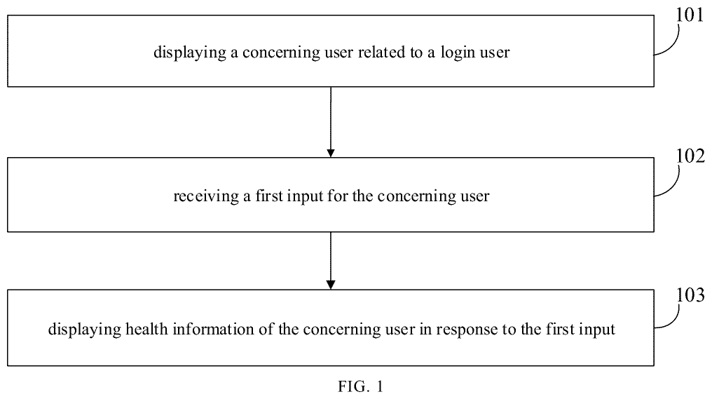

FIG. 1 schematically shows a flow chart of an information acquisition method provided by the present disclosure. An execution subject of the method may be any electronic equipment, for example, the method may be applied to an application with functions such as information displaying, information transmission and data processing and other functions, the method may be executed by a sever or terminal equipment of the application. Optionally, the method may be executed by the terminal equipment, the method includes:

Step 101, displaying a concerning user related to a login user.

In some embodiments of the present disclosure, the login user refers to a user who completes a login process in a client of the application, the login user may perform a registration process in advance through the client of the application, and then perform the login process through account information obtained by the registration process. The concerning user refers to a user related to the login user, an association relationship between the login user and the concerning user may be set by the login user himself, or the relationship may be automatically set by a system of the application according to user information of the login user and the concerning user. Establishment of the association relationship may be set by practical needs and will not be limited herein.

In some embodiments of the present disclosure, the login user may be a guardian who needs to monitor persons under guardianship such as an elderly, a child, a patient, a disabled person, and the concerning user may be the person under guardianship who needs to be monitored by the guardian. The guardian may establish the association relationship with the person under guardianship as the login user through the application provided by some embodiments of the present disclosure, to make the person under guardianship become the person being followed related to the login user. Certainly, this is only an example illustrated here, as long as the user who uses the application client provided by the embodiment of the present disclosure may become the login user or the concerning user, and the user who does not use the application client may become the concerning user either, the specific setting may be made according to practical needs which will not be limited herein.

Step 102, receiving a first input for the concerning user.

In the embodiment of the present disclosure, the first input may be an input of the user such as clicking, long pressing, sliding, voice, gesture and so forth of the user which are displayed in an application interface of the application client, the specific setting may be made according to practical needs which will not be limited herein.

In some embodiments of the present disclosure, the application client may respond to the first input of the user, which is corresponding to the user identification of the concerning user shown in the application interface, to determine the concerning user whose health information wanted to be viewed by the user. The concerning user may be one or more users concerning user except for the login user, and the concerning user may also be the concerning user himself so that the concerning user may view his health information through the application client.

Step 103, displaying health information of the concerning user in response to the first input.

In the embodiment of the present disclosure, the application client may determine the user being following corresponding to the user identification which is indicated by the first input, and then the application client may acquire the health information of the concerning user from local data resources or remote data resources such as background servers and cloud severs and so forth, to display the health information of the concerning user in the current interface of the client or by jumping to other interfaces, so that the user may view the health information of the concerning user in a display screen of terminal equipment.

In some embodiments of the present disclosure, the health information may be physical indicators information obtained by detecting physical signs of the concerning user through user monitoring equipment, the health information may also be user activity information obtained by monitoring activity ways of the concerning user, and the health information may also be user behavior trajectories obtained by detecting behavior trajectories of the concerning user, user mood information may be obtained by detecting and analyzing expression actions of the concerning user through image acquisition equipment. The specific content of the health information may be set according to practical needs which will not be limited herein. It should be noted that the health information is collected and provided to the login user after obtaining a consent of the concerning user or his guardian.

The embodiment of the present disclosure displays the health information of the concerning user according to a selection of the user, so that the user may view the health information of the concerning user conveniently.

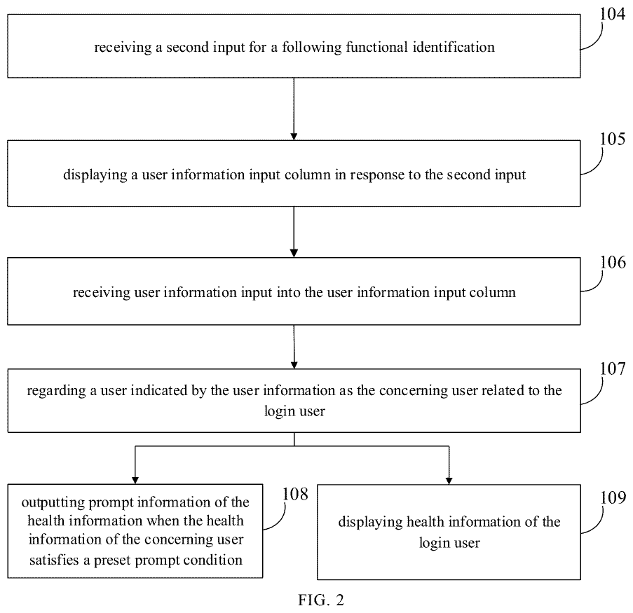

Optionally, referring to FIG. 2, the method further includes:

Step 104, receiving a second input for a following functional identification.

In the embodiment of the present disclosure, the following functional identification is configured to trigger an addition of a functional identification of the concerning user, the second input may be an input such as clicking, long pressing, sliding, voice, gesture and so forth for the following functional identification.

In some embodiments of the present disclosure, the application client may display the following functional identification as an entrance for the user adding the concerning user through the application interface of the client. Specifically, if the login user wants to add a new concerning user, a process of adding the concerning user may be triggered by the second input of the following functional identification in the application interface.

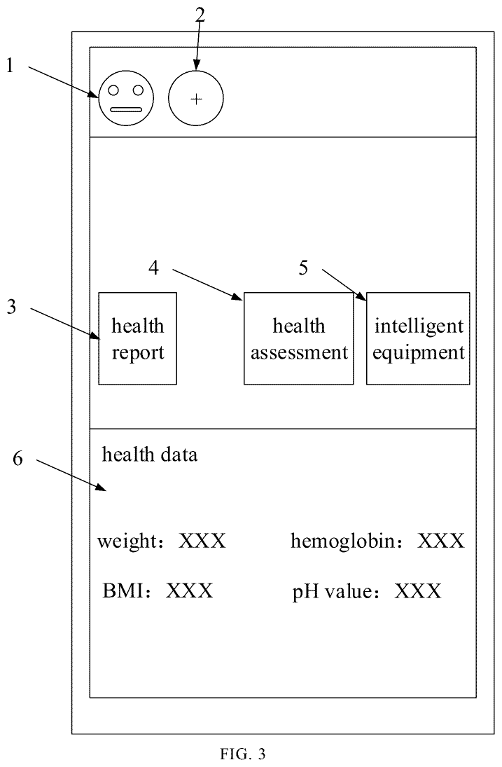

For example, referring to FIG. 3, which shows an schematic diagram showing the effect of an application interface provided by the embodiment of the present disclosure, the figure includes a user profile picture 1, a following functional identification 2, a health report functional identification 3, a health assessment functional identification 4, intelligent equipment functional identification 5 and a health data displaying column 6. The user may trigger the process of adding the concerning user by input ways such as clicking and long pressing the following functional identification 2.

Step 105, displaying a user information input column in response to the second input.

In the embodiment of the present disclosure, the user information input column is an input area for the user to add user information. A surrounding area or a background section of the user information input column may display prompt information of the user information needed to be filled such as category requirements, data volume requirements and matters that are used to prompt the user to fill in the user information column, so that the user may easily aware of what to fill in the user information input column.

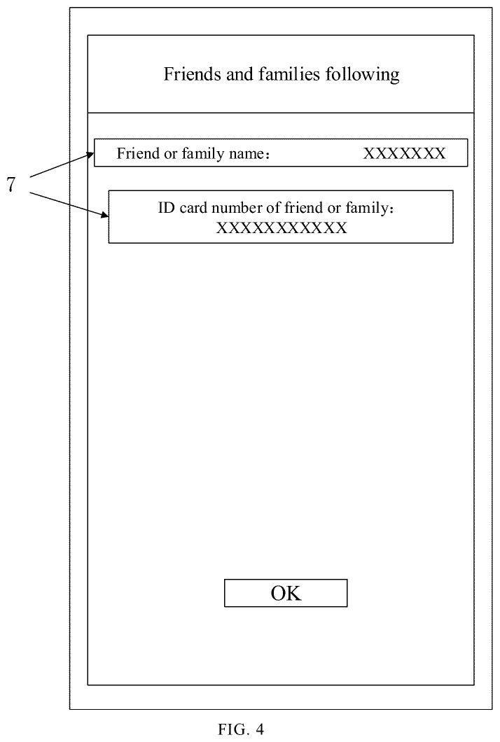

For example, referring to FIG. 4, in some embodiments of the present disclosure, the user information input column may be displayed by the application interface followed by families and friends, the user information column 7 is a name of a family or a friend and his ID card number. Therefore, on left sides of two user information input columns display prompt information of a name of ‘a family or a friend’ and ‘ID card number’, and prompt information of ‘please fill in a real name’ and ‘please input an ID card number’ are also marked in the user information input column, so that the user may intuitively and conveniently know where is the user information column and how to fill it. Certainly, this is only an example illustrated here. The specific setting of the user information input column may be determined according to practical needs which will not be limited herein.

Step 106, receiving user information input into the user information input column.

In an embodiment of the present disclosure, the client of the application may regard information received from the user information input column as user information filled by the user.

Step 107, regarding a user indicated by the user information as the concerning user related to the login user.

In an embodiment of the present disclosure, a sever of the application may store a corresponding relationship between the user information and the user in advance, so that the client of the application may request the server to query which user is indicated by the user information. After the client knows the user indicated by the user information, the client may establish the association relationship between the login user and the user, so that the user indicated by the user information becomes the concerning user of the login user.

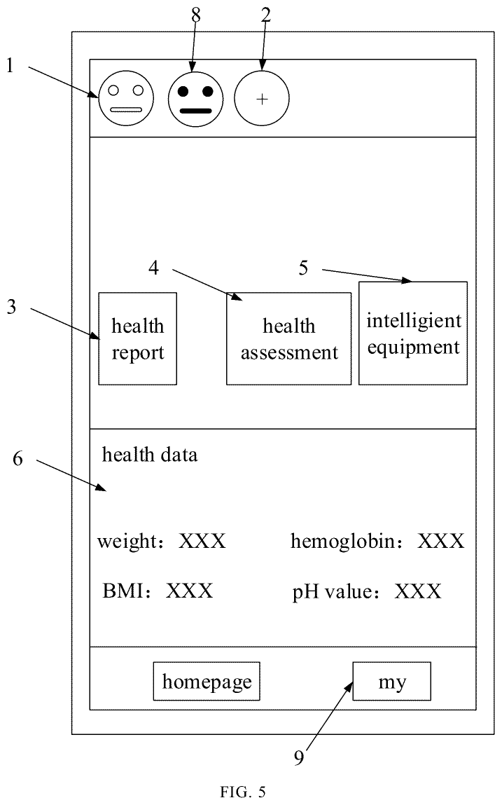

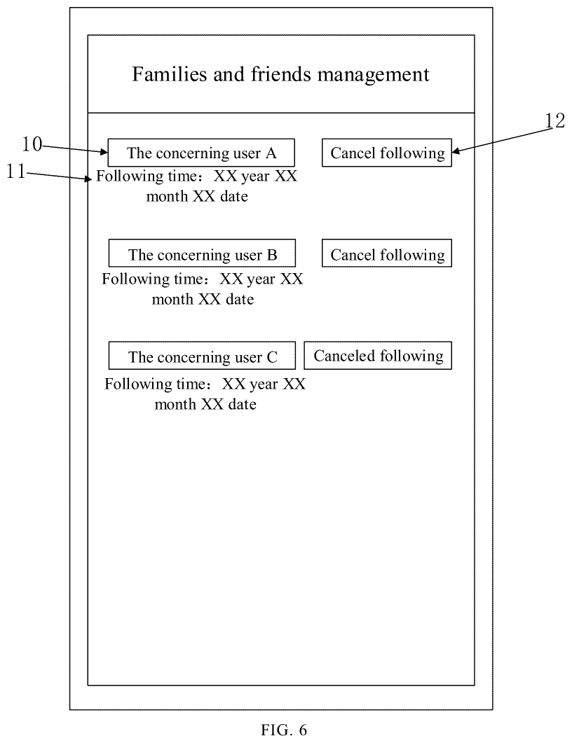

For example, referring to FIG. 5, after establishing the association relationship between the login user and the concerning user, the right side of the user profile picture 1 may display the concerning user profile picture 8 in the application interface of the client, and the user may also select the input through a ‘my’ functional identification 9 in the right lower corner of the application interface, to switch to the application interface shown in FIG. 6, which displays a list of related users of the login user, including a name 10 of the concerning user, a time of establishing the relationship 11 and a ‘cancel following’ functional identification 12 corresponding to each of the users being followed, the user may select and input via the functional identification 12 to delete the association relationship between the login user and the concerning user, for example, the concerning user C is a canceled association relationship, then prompt information of ‘the following relationship is canceled’ is displayed on the right side of the concerning user C.

Step 108, outputting prompt information of the health information when the health information of the concerning user satisfies a preset prompt condition.

In an embodiment of the present disclosure, the preset prompt condition refers to a condition that the health information is actively sent to the user to display what the health information needs to satisfy. The preset prompt condition may refer to a user indicated by the health information as a specific user, so that the user may view the health information of the specific user in time. The preset prompt condition may also be a specific content included by the health information, so that the user may view the specific content such as a health monitoring result of the concerning user in time. The preset prompt condition may also be all the health information received by the application client, which means when receiving the health information sent by the server, the prompt information may be outputted, so that the user may view the health information of the concerning user in time. The specific setting of the preset prompt condition may be set according to practical needs which will not be limited herein.

Step 109, displaying health information of the login user.

In an embodiment of the present disclosure, referring to FIG. 3, the health information of the login user may be default displayed in a health data displaying column 6. After the user operates to view the concerning user, the health information of the login user displayed in the health data displaying column may be changed to the health information of the concerning user, so that the user may view his own health information conveniently through the client of the application.

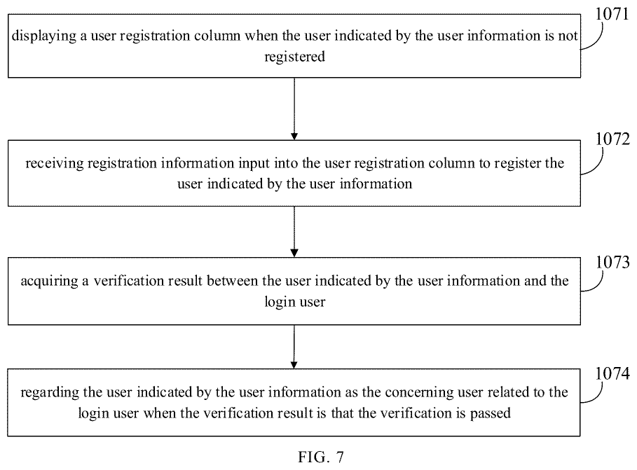

Optionally, referring to FIG. 7, the step 107 includes:
sub-step 1071, displaying a user registration column when the user indicated by the user information is not registered.

In an embodiment of the present disclosure, the client of the application transmits the user information to the server of the application, if the user indicated by the user information is not found, then it indicates that the user indicated by the user information is not registered, then the client of the application regards what is returned by the server as a retuning result of the registration, and displays the user registration column in the application interface to let the user register. It should be noted that due to the concerning user may be an elderly person, a child, a patients, a disable person and such groups who are inconvenient to use terminal equipment, therefore, a registration process may not be completed on the application by themselves, at this time, it may display the user registration column in the client of the login user to let the login user to complete the registration for the user he wants to follow in the application, it is not necessary for the concerning user himself to complete the registration process in the application, thereby improving the flexibility of application registration.

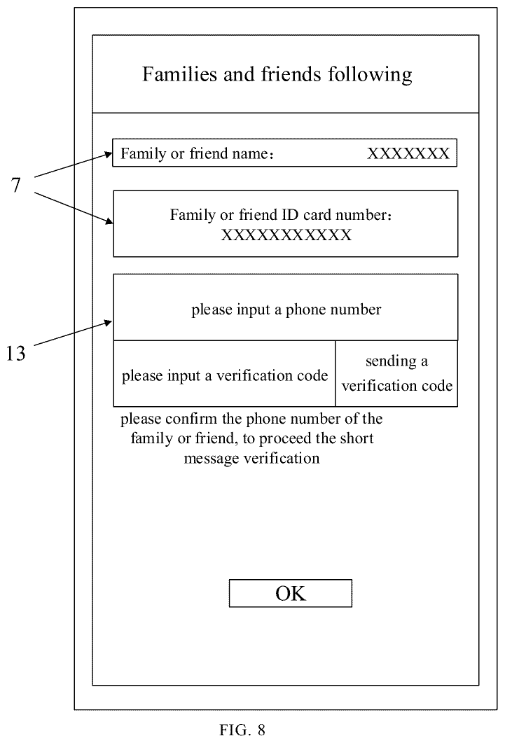

For example, referring to FIG. 8, the application interface of ‘families and friends following’ may further include a user registration column 13, the user registration column 13 includes prompt information 14 of ‘please input a phone number of a family or a friend’ and ‘please input a verification code’, and a functional identification 15 of ‘sending a verification code’, so that the user may select and input through the functional identification 15 to obtain a short message with the verification code to input the verification code in the user registration column.
sub-step 1072, receiving registration information input into the user registration column to register the user indicated by the user information.

In an embodiment of the present disclosure, the client of the application sends a user registration request to the sever of the application according to the registration information received in the user registration column, the server of the application registers the user indicated by the user information according to the registration information, and returns a successful registration notification information to the client after the registration is completed.
sub-step 1073, acquiring a verification result between the user indicated by the user information and the login user.

In some embodiments of the present disclosure, the client of the application may send an identification request carrying the user information to the server, the sever may generate a verification result by querying whether the user information in the user database matches a pre-stored standard user information. If the information is matched, the verification result is passed: if the information is not matched, the verification result is not passed, then the sever sends the verification result to the client.
sub-step 1074, regarding the user indicated by the user information as the concerning user related to the login user when the verification result is that the verification is passed.

In an embodiment of the present disclosure, then the verification result received by the client of the application is passed, then the association relationship between the login user and the concerning user may be established, and displays the prompt information of successful following to inform the user. In the contrary, if the verification result is not passed, then displays the prompt information of failure following to inform the user as well. It should be noted that, the sever may also establish the association relationship between the login user and the concerning user and store the association relationship, and send the established association relationship to the client, so that the client does not need to establish the association relationship repeatedly. The embodiment of the present disclosure establishes the association relationship between the login user and the concerning user, after the user information verification is passed, to ensure the accuracy of the establishment of the association relationship.

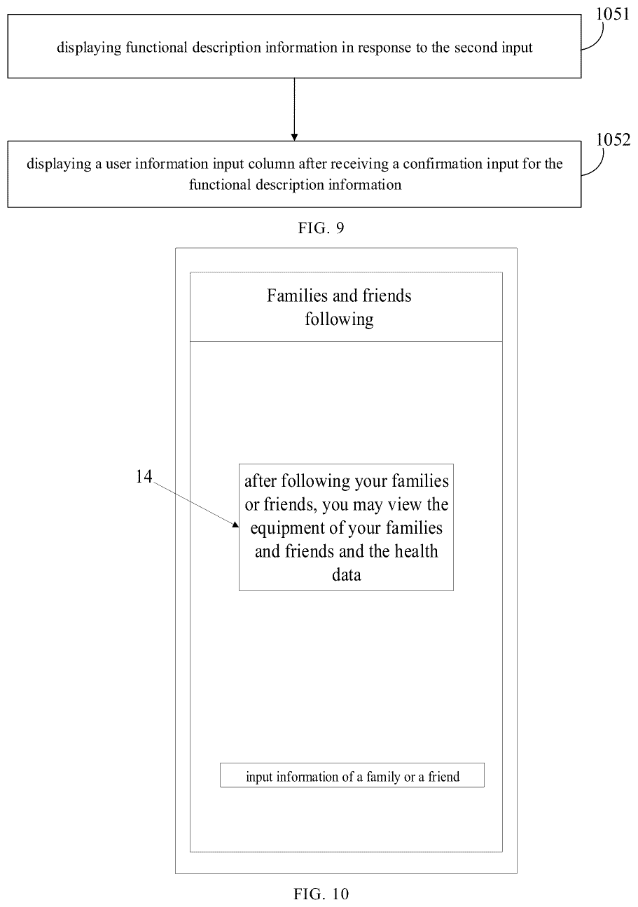

Optionally, referring to FIG. 9, the step 105 includes:
sub-step 1051, displaying functional description information in response to the second input.

In an embodiment of the present disclosure, the functional description information is used to inform the user that he may obtain description information of service functions after establishing the association relationship between the user and the concerning user, so that the user may obtain details of a function of user follows conveniently.
sub-step 1052, displaying a user information input column after receiving a confirmation input for the functional description information.

In an embodiment of the present disclosure, after the client of the application receives the confirmation input for the functional description information, then it is determined that the user is aware of the description information of the function of user follows to further show that the user information input column is configured for the user to establish a bonding relationship between the login user and the concerning user.

For example, referring to FIG. 10, a displaying column 16 including the functional description information may display the functional description information of ‘after following your families or friends, you may view the equipment of your families and friends and the health data’, the user may click a functional identification 17 of ‘input information of a family or a friend’ to confirm the input.

The embodiment of the present disclosure displays the functional description information for the user to view before executing the user follows process, and executes the subsequent user follows process after receiving the user confirmation input, so that the user may obtain the details of the user follows function conveniently.

Optionally, the step 108 may include: outputting the prompt information of the health information by a target prompt way, the target prompt way at least includes one of the followings: a short message prompt way, an email prompt way, a communication application prompt way, an audio prompt way, a video prompt way, a lighting prompt way, or a vibration way.

In some embodiments of the present disclosure, the client of the application may prompt the user to view the health information by the short message prompt way, the email prompt way, the communication application prompt way, the audio prompt way, the video prompt way, the lighting prompt way, or the vibration way, so that the user may view the health information of the concerning user in time. Certainly, there are other prompt ways with prompt functions that may suitable for the embodiment of the present disclosure, the specific way may be set according to practical needs which will not be limited herein.

Optionally, when the preset prompt condition is a behavior-trajectory abnormality condition, the health information include: behavior-trajectory information of the concerning user, the prompt information includes: behavior-trajectory abnormality prompt information, the step 108 may include: outputting the behavior-trajectory abnormality prompt information of the concerning user when the behavior-trajectory information of the concerning user satisfies the behavior-trajectory abnormality condition.

In an embodiment of the present disclosure, the behavior-trajectory abnormality condition refers to a condition that the health information should be met when the behavior and trajectory is abnormal of the concerning user. Specifically, it may be determined by behavior-trajectory information of the concerning user included in the health information which are the user behavior and trajectory. For example, when the distance between the location of the concerning user and the home location exceeds a distance threshold, or if the time of the concerning user leaves the home location exceeds a time threshold, then the behavior of the concerning user is determined as abnormal, then outputting trajectory abnormality prompt information such as ‘someone leaves home as violating the rule’ and ‘someone leaves far from home’. The embodiment of the present disclosure outputs the behavior-trajectory abnormality prompt information when the behavior-trajectory information of the concerning user satisfies the behavior-trajectory abnormality prompt condition, to make the user know the behavior-trajectory abnormality situation of the concerning user in time.

Optionally, when the preset prompt condition is a health abnormality condition, the health information includes: the physical indicators of the concerning user, the prompt information includes: health abnormality prompt information, the step 108 may include: outputting the health abnormality prompt information of the concerning user when the physical indicators of the concerning user satisfy the health abnormality condition.

In an embodiment of the present disclosure, the health abnormality condition refers to a condition that the health information should be met when the physical indicators of the concerning user are abnormal. Specifically, it may be determined by health indicators of the concerning user included in the health information. For example, determining the health of the concerning user is abnormal when a heart rate, a blood pressure, a blood lipid and so forth exceeds physical indicator thresholds, then outputting the health abnormality prompt information such as ‘someone's heart rate is low.’ ‘someone's blood pressure is high’ and so forth. The present disclosure outputs the health abnormality prompt information when the physical indicators of the concerning user satisfy the health abnormality prompt condition, so that the user may be informed the health abnormality situation of the concerning user in time.

Optionally, when the preset prompt condition is a diagnosis and treatment abnormality condition, the health information includes: a medication taking record and a physical examination record of the concerning user, the prompt information includes: diagnosis and treatment abnormality prompt information, the step 108 may include: outputting the diagnosis and treatment abnormality prompt information of the concerning user when the medication taking record and the physical examination record of the concerning user satisfy the diagnosis and treatment abnormality condition.

In an embodiment of the present disclosure, the diagnosis and treatment abnormality condition refer to a condition that the health information should be met when the medication taking record and the physical examination record are abnormal. Specifically, it may be determined by the medication taking record and the physical examination record of the concerning user included in the health information. For example: a drug reuse measurement and time of the concerning user is inconsistent with a drug reuse measurement and recommended taking time in a medical order, or the concerning user fails to do a health monitoring according to the medical order, then outputting the diagnosis and treatment abnormality prompt information such as ‘someone does not take medicine on time’ and ‘someone does not do physical examination on time’. The present disclosure outputs the diagnosis and treatment abnormality prompt information when the physical indicators of the concerning user satisfy the diagnosis and treatment abnormality prompt condition, so that the user may be informed the diagnosis and treatment abnormality situation of the concerning user in time.

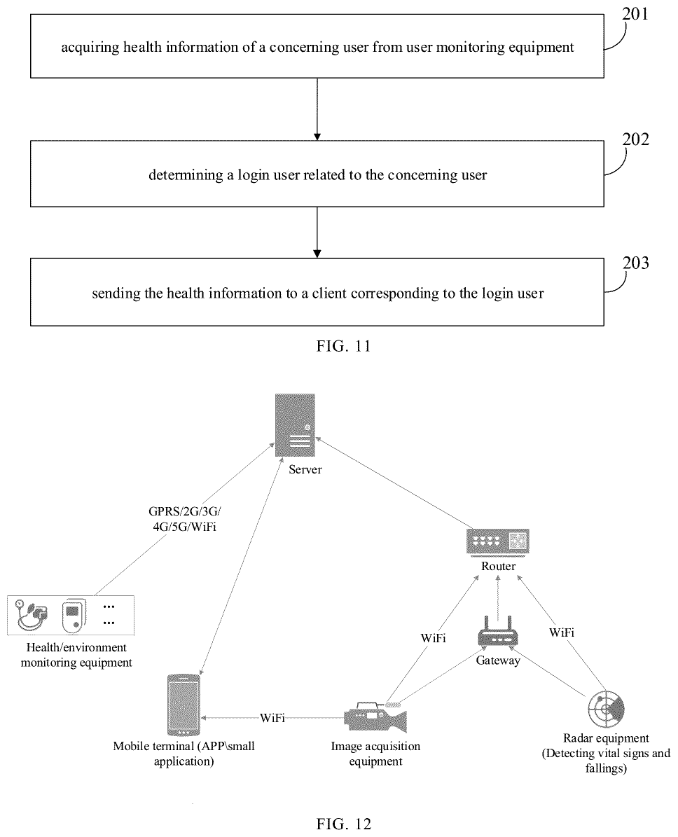

FIG. 11 schematically shows a flow chart of an information providing method provided by the present disclosure. An execution subject of the method may be any electronic equipment, for example, the method may be applied to an application with functions such as information displaying, information transmission and data processing, the method may be executed by a sever or terminal equipment of the application, optionally, the method may be executed by the sever, the method includes:

Step 201, acquiring health information of a concerning user from user monitoring equipment.

In an embodiment of the present disclosure, the user monitoring equipment may be electronic equipment connected with the server for monitoring daily living and physical indicators of the user and other related health information, and sending the monitored data to the server. For example, according to FIG. 12, the user detection equipment may be the health monitoring equipment of the Internet of Things. Through GPRS/2G/3G/4G/5G/WIFI and other ways, the Internet of Things technology is configured to directly establish communication with the server, to ensure the collected data are real and effective. Radar equipment may also be configured to monitor vital signs and falling situation of a person at home, and establish communication with the server through gateways or WIFI. It may also be image acquisition equipment for monitoring overall activities of an elderly person at home and whether he leaves home. The sever may provide user information collected by every user monitoring data to a client of an application of a mobile terminal. The specific user monitoring equipment may be set according to practical needs which will not be limited herein.

Step 202, determining a login user related to the concerning user.

In an embodiment of the present disclosure, the sever may obtain the login user related to the concerning user by querying a pre-stored relationship between the concerning user and the login user in a user database.

Step 203, sending the health information to a client corresponding to the login user.

In an embodiment of the present disclosure, the sever may send the health information of the concerning user to the client regularly, or the sever may send the health information to the client when the client requests to obtain the health information.

In the embodiment of the present disclosure, by sending the health information to the client where the login user related to the concerning user at to display, so that the user may view the health information of the concerning user conveniently.

Therefore, the user may view the health information of the concerning user conveniently through the client.

Optionally, referring to FIG. 13, the method further includes:

Step 204, receiving user information sent by the client corresponding to the login user.

In an embodiment of the present disclosure, the server receives a user following request with the user information from the client, and starts to execute a user following process between the login user and the concerning user.

Step 205, acquiring a trust value between a user indicated by the user information and the login user according to the user information.

In an embodiment of the present disclosure, the trust value is a trust degree between the login user and the user indicated by the user information, specifically, the trust value may be determined by a difference between the user information and standard information of the user indicated by the user information. The standard information is included in the server in advance when the user is registered. It should be noted that if the difference between the standard information of the login user and the user indicated by the user information is smaller, it means that the trust value between the login user and the user is larger. On the contrary, if the difference is larger, then it means that the trust value between the login user and the user is smaller.

Step 206, when the trust value is greater than a trust value threshold, determining a verification result between the user indicated by the user information and the login user is passed, and sending the verification result to the client.

In an embodiment of the present disclosure, the trust value threshold may be set by the user himself or default set by the system. When the trust value is greater than the trust value threshold, it means that the trust value between the user indicated by the user information and the login user meets a requirement of establishing the association relationship, and then the verification result is sent to the client as passed.

The embodiment of the present disclosure, by outputting the verification result as the verification is passed when the trust value between the users is greater than the trust value threshold, and establishing the association relationship between the login user and the concerning user is allowed after, which avoids a user without a trust relationship with the concerning user to acquire the health information of the concerning user and ensures the safety of establishing the association relationship.

Step 207, establishing a following relationship between a target user account, the user indicated by the user information and an anonymous user, a user attribute of the anonymous user is the same as the user indicated by the user information.

In an embodiment of the present disclosure, by generating a plurality of anonymous users with a same attribute as the concerning user, the login user establishes pseudo association relationships with the plurality of anonymous users, and establishes the association relationship between the login user and the concerning user. Then, the association relationship with the concerning user and the plurality of pseudo association relationships are summarized and hidden, so that the login user has the plurality of pseudo association relationships which are the same as the user information of the concerning user, so that an attack to any of the association relationships may be transferred to the plurality of pseudo relationships, and the probability of finding the real target will be greatly reduced.

For example, if the quantity of generated anonymous users is m, so that each of the association relationships at least has the same user attribute as k−1 associated records, so that the probability of finding the real association relationship is 1/(m+k). By protecting the user relationship, it is ensured that a relationship privacy between the users may be protected while both users with established family relationship enjoy a quality service provided by the system, and other data of the users will not be leaked due to malicious attacks to the user relationship.

Optionally, the user information at least includes: user identification information, user reply information and the user question quantity, referring to FIG. 14, the step 205 includes:
sub-step 2051, querying a user indicated by the user information according to the user identification information.

In an embodiment of the present disclosure, the user identification information may be information such as name, gender, age, and ID card number that may represent a user's user identification. The sever may query the user indicated by the user information in the user database according to the user identification information.
sub-step 2052, acquiring the trust value between the user indicated by the user information and the login user according to the user reply information and the user question quantity.

In an embodiment of the present disclosure, the user reply information is the reply information that the user inputs on the client for the user question. The user question is a question configured to verify whether the login user and the user indicated by the user information trust each other. Specifically, it may be set by the user himself, or it may be automatically set by the system according to the user database, for example the user question may be ‘what is the hobby of someone’, ‘who is the middle school teacher of someone’ and ‘which school is someone graduated from’ and so forth. These questions related to life experiences of the user are usually only known by people with higher trust relationships. Therefore, the trust value between the login user and the user indicated by the user information may be calculated by the user reply information for the user questions and the user question quantity.

Optionally, the sub-step 2052 includes:

According to the user reply information and the user question quantity, acquiring the trust value between the user indicated by the user information and the login user through following formulas:

$$\begin{matrix}T = E ( Beta ( \begin{matrix}θ \vert r + 1 , & s + 1\end{matrix} ) ) = \frac{r + 1}{r + s + 2} \\ r = r 1 + r 2\end{matrix}$$

wherein, the T is the trust value between the user indicated by the user information and the login user, the r1 is a quantity of questions matched between the user reply information and standard question-answer information, the r2 is a sum of probability matched between the user reply information and the standard question-answer information, the s is a quantity of questions not matched between the user reply information and the standard question-answer information.

In an embodiment of the present disclosure, the sever may acquire a matching probability according to the similarity between the user reply information and standard question-answer information, and by setting a probability threshold, to determine the matching between the user reply information and the standard question-answer information when the matching probability between the user reply information and the standard question-answer information is greater than the probability threshold. Furthermore, confusion questions may also be set in the user questions which are not related to the user's real life to let the user to reply, to further improve the accuracy of trust verification.

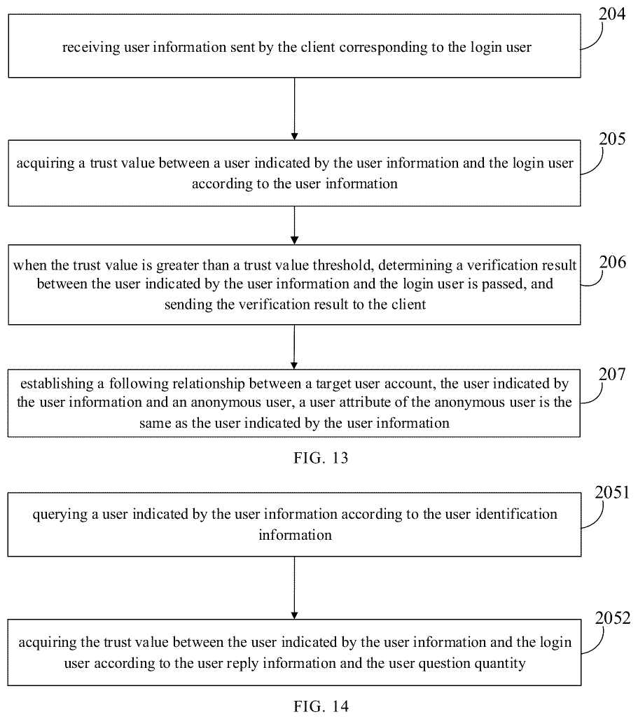

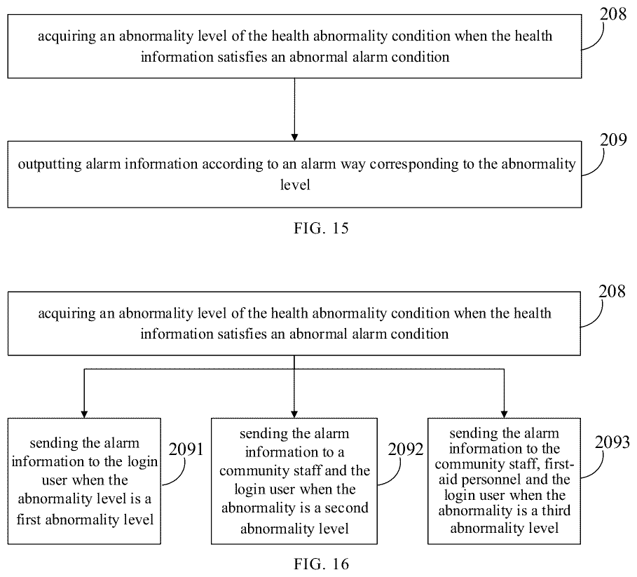

Optionally, referring to FIG. 15, the method further includes:

Step 208, acquiring an abnormality level of the health abnormality condition when the health information satisfies an abnormal alarm condition.

In an embodiment of the present disclosure, the abnormal alarm condition refers to a condition that the health information should be met when the server outputs alarm information, for example, the abnormal alarm condition may reflect the health information of the elderly falls, stops breathing and other emergencies. Different abnormal alarm conditions may establish corresponding relationships with abnormality levels in advance and be stored in the sever.

Step 209, outputting alarm information according to an alarm way corresponding to the abnormality level.

In an embodiment of the present disclosure, different abnormality levels correspond to different alarm ways. For example, outputting alarm information by one alarm way when the abnormality level is low, or outputting alarm information by a plurality of alarm ways when the abnormality level is high. Certainly, different alarm ways for different abnormality levels may be set according to practical needs which will not be limited herein. The alarm information refers to the prompt information of matters reflected by prompt health information of the user, for example, the alarm information may be ‘someone is in urgent need of rescue’ and ‘someone is out of touch’ and so forth, so that a receiver of the alarm information may provide helps to the concerning user in time. The embodiment of the present disclosure, by outputting alarm according to the alarm way corresponding to the abnormality condition satisfied by the health information, so that the concerning user may send the alarm information when he meets an emergency, thereby improving the safety of user monitoring.

Optionally, referring to FIG. 16, the step 209 includes:
sub-step 2091, sending the alarm information to the login user when the abnormality level is a first abnormality level.
sub-step 2092, sending the alarm information to a community staff and the login user when the abnormality is a second abnormality level.
sub-step 2093, sending the alarm information to the community staff, first-aid personnel and the login user when the abnormality is a third abnormality level.

In an embodiment of the present disclosure, the first abnormality level, the second abnormality level and the third abnormality level are the three abnormality levels which the emergency degree increase in turn. Therefore, when it is the first abnormality level, it may only send the alarm information and synchronize the abnormal health information to the login user related to the concerning user: when it is the second abnormality level, it may send the alarm information to the community staff and the login user at the same time, so that when the login user may not reach to the concerning user to provide assistance in time, then the community staff may provide assistance to the concerning user; and

When it is the third abnormality level, it may directly send the alarm information to the first-aid personnel of a hospital, the login user and the community staff at the same time, it may also send the health information and medical history of the concerning user, an emergency contact, a family location and other related information to the first-aid personnel, so that the first-aid personnel may reach to the concerning user to provide rescue in time. For example, items reflected by the health information of the first abnormality level may be an item with a low safety risk such as the concerning user does not return to home, items reflected by the health information of the second abnormality level may be an item with a middle safety risk such as the concerning user falls and other things that need helps from others, items reflected by the health information of the third abnormality level may be an item with a high safety risk such as the concerning user stops breathing, vital signs are weak and so forth. Specifically, the abnormality level and the corresponding alarm way may be set according to practical needs which will not be limited here. The embodiment of the present disclosure may choose to send the alarm information to the login user, the community staff, and the first-aid personnel according to different abnormality levels, so that the concerning user may get helps when he is in danger, thereby improving the safety of the user monitoring.

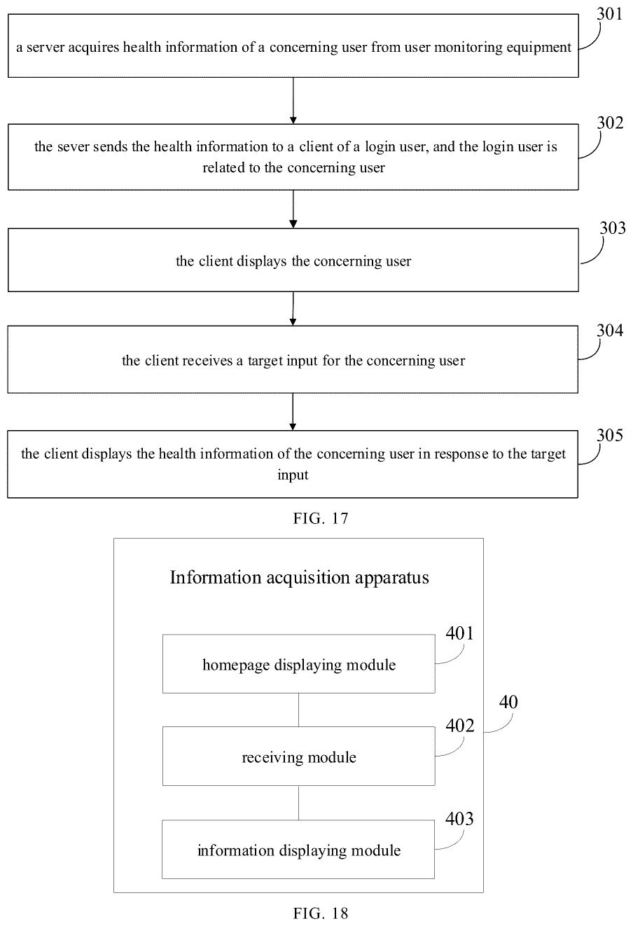

FIG. 17 schematically shows a flow chart of an information interaction method provided by the present disclosure, and the method includes:

Step 301, a server acquires health information of a concerning user from user monitoring equipment.

The step may be referred to the detailed description of step 201 which will not be repeated herein.

Step 302, the sever sends the health information to a client of a login user, and the login user is related to the concerning user.

The step may be referred to the detailed description of step 203 which will not be repeated herein.

Step 303, the client displays the concerning user.

The step may be referred to the detailed description of step 101 which will not be repeated herein.

Step 304, the client receives a target input for the concerning user.

The step may be referred to the detailed description of step 102 which will not be repeated herein. It should be noted that, the target input is similar to the first input in step 102, which are both configured to select the concerning user.

Step 305, the client displays the health information of the concerning user in response to the target input.

The step may be referred to the detailed description of step 103 which will not be repeated herein.

An embodiment of the present disclosure displays the health information according to the concerning user selected by the user, so that the user may view the health information of the concerning user conveniently.

FIG. 18 schematically shows a structural schematic diagram of an information acquisition apparatus 40 provided by the present disclosure, and the apparatus includes:
a homepage displaying module 401 configured for displaying a concerning user related to a login user;
a receiving module 402 configured for receiving a first input for the concerning user;
an information displaying module 403 configured for displaying health information of the concerning user in response to the first input.

Optionally, the apparatus further includes:
an association module is configured for:
receiving a second input for a following functional identification;
displaying a user information input column in response to the second input;
receiving user information input into the user information input column
regarding a user indicated by the user information as the concerning user related to the login user.

Optionally, the association module is further configured for:
acquiring a verification result between the user indicated by the user information and the login user;
when the verification result is that the verification is passed, regarding the user indicated by the user information as the concerning user related to the login user.

Optionally, the association module is further configured for:
displaying a user registration column when the user indicated by the user information is not registered;
receiving registration information input into the user registration column to register the user indicated by the user information.

Optionally, the association module is further configured for:
displaying functional description information in response to the second input;
displaying a user information input column after receiving a confirmation input for the functional description information.

Optionally, the information displaying module 403 is further configured for:
outputting prompt information of the health information when the health information of the concerning user satisfies a preset prompt condition.

Optionally, the information displaying module 403 is further configured for:
outputting the prompt information of the health information by a target prompt way, the target prompt way at least includes one of the followings: a short message prompt way, an email prompt way, a communication application prompt way, an audio prompt way, a video prompt way, a lighting prompt way, or a vibration way.

Optionally, when the preset prompt condition is a behavior-trajectory abnormality condition, the health information includes: behavior-trajectory information of the concerning user, the prompt information includes: behavior-trajectory abnormality prompt information:

The information displaying module 403 is further configured for:
outputting the behavior-trajectory abnormality prompt information of the concerning user when the behavior-trajectory information of the concerning user satisfies the behavior-trajectory abnormality condition.

Optionally, when the preset prompt condition is a health abnormality condition, the health information includes: the physical indicators of the concerning user, the prompt information includes: health abnormality prompt information;

The information displaying module 403 is further configured for:
outputting the health abnormality prompt information of the concerning user when the physical indicators of the concerning user satisfy the health abnormality condition.

Optionally, when the preset prompt condition is a diagnosis and treatment abnormality condition, the health information includes: a medication taking record and a physical examination record of the concerning user, the prompt information includes: diagnosis and treatment abnormality prompt information:

The information displaying module 403 is further configured for:
outputting the diagnosis and treatment abnormality prompt information of the concerning user when the medication taking record and the physical examination record of the concerning user satisfy the diagnosis and treatment abnormality condition.

Optionally, the homepage displaying module 401 is further configured for:
displaying health information of the login user.

The embodiment of the present disclosure displays the health information of the concerning user selected by the user, so that the user may view the health information of the concerning user conveniently.

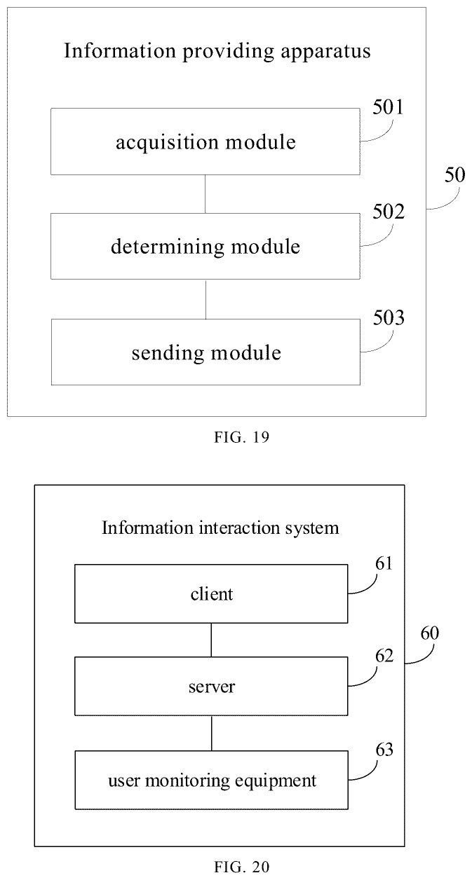

FIG. 19 schematically shows a structural schematic diagram of an information providing apparatus 50 provided by the present disclosure, and the apparatus includes:
an acquisition module 501 configured for acquiring health information of a concerning user from user monitoring equipment;
a determining module 502 configured for determining a login user related to the concerning user;
a sending module 503 configured for sending the health information to a client corresponding to the login user.

Optionally, the apparatus further includes:
a verification module is configured for:
receiving user information sent from the client corresponding to the login user;
acquiring a trust value between a user indicated by the user information and the login user according to the user information;
when the trust value is greater than a trust value threshold, determining a verification result between the user indicated by the user information and the login user is passed, and sending the verification result to the client.

Optionally, the user information at least includes: user identification information, user reply information and the user question quantity:

The verification module is further configured for:
querying a user indicated by the user information according to the user identification information;
acquiring the trust value between the user indicated by the user information and the login user according to the user reply information and the user question quantity.

Optionally, the verification module is further configured for:
according to the user reply information and the user question quantity, acquiring the trust value between the user indicated by the user information and the login user through following formulas:

$$\begin{matrix}T = E ( Beta ( \begin{matrix}θ \vert r + 1 , & s + 1\end{matrix} ) ) = \frac{r + 1}{r + s + 2} \\ r = r 1 + r 2\end{matrix}$$

wherein, the T is the trust value between the user indicated by the user information and the login user, the r1 is a quantity of questions matched between the user reply information and standard question-answer information, the r2 is a sum of probability matched between the user reply information and the standard question-answer information, the s is a quantity of questions not matched between the user reply information and the standard question-answer information.

Optionally, the verification module is further configured for:
establishing a following relationship between a target user account, the user indicated by the user information and an anonymous user, a user attribute of the anonymous user is the same as the user indicated by the user information.

Optionally, the apparatus further includes:
an alarm module is configured for:
acquiring an abnormality level of the health abnormality condition when the health information satisfies an abnormal alarm condition;
outputting alarm information according to an alarm way corresponding to the abnormality level.

Optionally, the alarm module is further configured for:
sending the alarm information to the login user when the abnormality level is a first abnormality level;
sending the alarm information to a community staff and the login user when the abnormality level is a second abnormality level;
sending the alarm information to the community staff, first-aid personnel, and the login user when the abnormality level is a third abnormality level.

In the embodiment of the present disclosure, by sending the health information to the client where the login user related to the concerning user at to display, so that the user may view the health information of the concerning user conveniently.

FIG. 20 schematically shows a structural schematic diagram of an information interaction system 60 provided by the present disclosure, the system includes: a client 61, a server 62 and monitoring equipment 63:

The server 62 is configured for:
acquiring health information of a concerning user from the user monitoring equipment 63;
sending the health information to a client of a login user, the login user is related to the concerning user;

The client 61 is configured for:
displaying the concerning user;
receiving a target input for the concerning user;
displaying the health information of the concerning user in response to the target input.

The embodiment of the apparatus described above is only schematic, wherein the components described separately may or may not physically apart, the component as the unit display may or may not be a physical component, which may be in one place or it may be distributed to a plurality of network units. Part of or all the modules may be selected to implement the purpose of the embodiment according to practical needs. Ordinary technical personnel in this field may understand and implement it without paying creative labor.

The individual component embodiments of this disclosure may be implemented in hardware, in software modules running on one or more processors, or in a combination of them. Those skilled in the field should understand that a microprocessor or digital signal processor (DSP) may be used in practice to implement some or all of the functions of some or all of the components of a computational processing device according to this disclosed embodiment. This exposure may also be implemented as devices or device programs (for example, computer programs and computer program products) used to perform some or all of the methods described here. Such implementation the program exposed may be stored on computer readable media or may take the form of one or more signals. Such signals may be downloaded from Internet sites, provided on carrier signals, or in any other form.

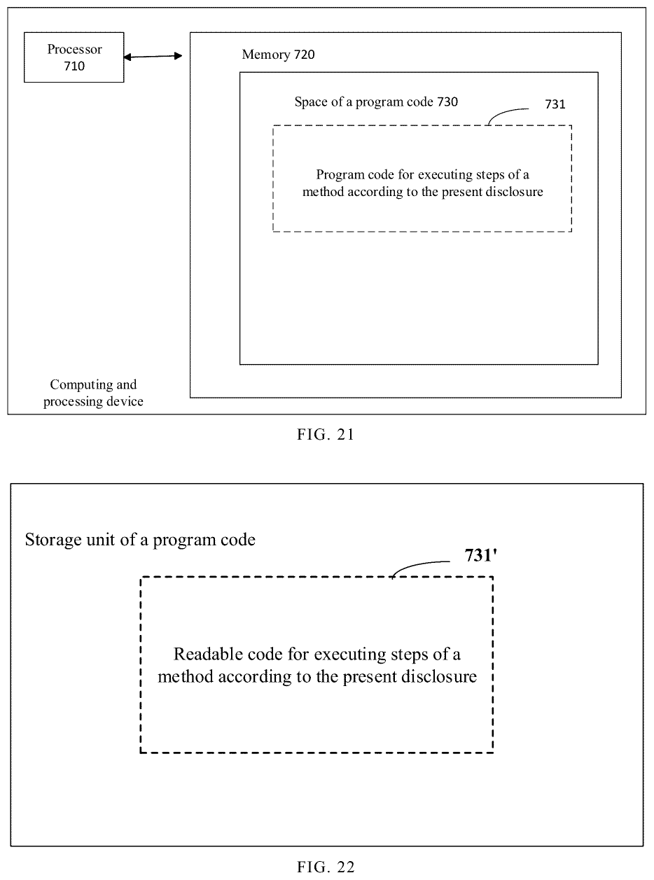

For example, FIG. 21 shows a computing and processing device that may be implemented according to the method provided by the present disclosure. The computing and processing device traditionally include a processor 710 and a computer program product or computer readable medium in the form of a memory 720. The Memory 720 may be an electronic memory such as flash memory. EEPROM (electrically erasable programmable Read-only memory), EPROM, hard disk, or ROM. Memory 720 has storage 730 of program code 731 for performing any of the method steps described above. For example, storage 730 for program code could include individual program code 731 for implementing the various steps in the method above. The program code may be read from or written to one or more computer program products. These computer program products include program code carriers such as hard disks, compact disks (CDS), memory cards, or floppy disks. Such computer program products are usually portable or stationary storage units as described in FIG. 22. The storage unit may have a storage segment, storage space, etc., arranged similarly to memory 720 in the computational processing device shown in FIG. 21. The program code may, for example, be compressed in an appropriate form. Typically, the storage unit includes computer-readable code 731′, that is, code that may be read by, for example, a processor such as 710, which, when executed by a computing and processing device, causes the compute processing device to perform the steps in the method described above.

It should be understood that the steps in the attached flow chart are shown in sequence as indicated by the arrows, but they are not necessarily carried out in the sequence indicated by the arrows. Unless explicitly stated in the description, there is no strict order in which these steps may be performed, and they are capable to be performed in any other sequences. Moreover, at least part of the steps in the attached flow chart may include multiple sub-steps or stages, these sub-steps or stages are not necessarily executed at the same time, but may be executed at different times, and their execution sequence is not necessarily sequential. It may be performed alternately with other steps or at least part of a sub-step or stage of another step.

The term ‘one embodiment’. ‘an embodiment’, or ‘one or more embodiments’ herein means that the particular features, structures, or features described in combination with embodiments are included in at least one embodiment disclosed herein. Also, note that the examples of words ‘in an embodiment’ here do not necessarily all refer to the same embodiment.

A great deal of detail is provided in the manual provided here. However, it is understood that this disclosed embodiment may be practiced without such specific details. In some instances, known methods, structures and techniques are not detailed so as not to obscure the understanding of this specification.

In a claim, no reference symbol between parentheses shall be constructed to restrict the claim. The word ‘include’ does not exclude the existence of elements or steps not listed in the claim. The word ‘one’ or ‘one’ before a component does not preclude the existence of more than one such component. This exposure may be implemented with the help of hardware including several different components and with the help of properly programmed computers. In listing the unit claims of several devices, several of these devices may be embodied by the same hardware item. The use of the words first, second, and third does not indicate any order. These words may be interpreted as names.

Finally, it should be noted that the above embodiments are only used to illustrate, and not to limit, the disclosed technical solution; notwithstanding the detailed description of this disclosure with reference to the foregoing embodiments, ordinary technical personnel in the field should understand that they may still modify the technical solutions recorded in the foregoing embodiments, or make equivalent substitutions to some of the technical features thereof; such modifications or substitutions shall not separate the essence of the corresponding technical solutions from the spirit and scope of the technical solutions of the disclosed embodiments.

1. An information acquisition method, wherein the method comprises:
displaying a concerning user related to a login user;
receiving a first input for the concerning user; and
displaying health information of the concerning user in response to the first input.

2. The method according to claim 1, wherein the method further comprises:
receiving a second input for a following functional identification;
displaying a user information input column in response to the second input;
receiving user information input into the user information input column; and
regarding a user indicated by the user information as the concerning user related to the login user.

3. The method according to claim 2, wherein regarding the user indicated by the user information as the concerning user related to the login user comprises:
acquiring a verification result between the user indicated by the user information and the login user; and
when the verification result is that the verification is passed, regarding the user indicated by the user information as the concerning user related to the login user.

4. The method according to claim 3, wherein before acquiring the verification result between the user indicated by the user information and the login user, the method further comprises:
displaying a user registration column when the user indicated by the user information is not registered; and
receiving registration information input into the user registration column to register the user indicated by the user information.

5. The method according to claim 2, wherein displaying the user information input column in response to the second input comprises:
displaying functional description information in response to the second input; and
displaying the user information input column after receiving a confirmation input of the functional description information.

6. The method according to claim 1, wherein the method further comprises:
outputting prompt information of the health information when the health information of the concerning user satisfies a preset prompt condition.

7. The method according to claim 6, wherein outputting the prompt information of the health information comprises:
outputting the prompt information of the health information in a target prompt way, the target prompt way at least comprises at least one of a short message prompt way, an email prompt way, a communication-application prompt way, an audio prompt way, a video prompt way, a light prompt way and a vibration prompt way.

8. The method according to claim 6, wherein when the preset prompt condition is a behavior-trajectory abnormality condition, the health information comprises: behavior-trajectory information of the concerning user, the prompt information comprises: a behavior-trajectory abnormality prompt information;
outputting the prompt information of the health information when the health information of the concerning user satisfies the preset prompt condition comprises:
outputting the behavior-trajectory abnormality prompt information of the concerning user when the behavior-trajectory information of the concerning user satisfies the behavior-trajectory abnormality condition.

9. The method according to claim 6, wherein when the preset prompt condition is a health abnormality condition, the health information comprises: physical indicators of the concerning user, the prompt information comprises: health abnormality prompt information;
outputting the prompt information of the health information when the health information of the concerning user satisfies the preset prompt condition comprises:
outputting the health abnormality prompt information of the concerning user when the physical indicators of the concerning user satisfy the health abnormality condition.

10. The method according to claim 6, wherein when the preset prompt condition is a diagnosis and treatment abnormality condition, the health information comprises: a drug taking record and a physical examination record of the concerning user, the prompt information comprises: a diagnosis and treatment abnormality prompt information;
outputting the prompt information of the health information when the health information of the concerning user satisfies the preset prompt condition comprises:
outputting the diagnosis and treatment abnormality prompt information of the concerning user when the drug taking record and the physical examination record of the concerning user satisfy the diagnosis and treatment abnormality condition.

11. The method according to claim 1, wherein the method further comprises:
displaying the health information of the login user.

12. An information providing method, wherein the method comprises:
acquiring health information of a concerning user from user monitoring equipment;
determining a login user related to the concerning user; and
sending the health information to a client corresponding to the login user.

13. The method according to claim 12, wherein the method further comprises:
receiving user information sent by the client corresponding to the login user;
acquiring a trust value between a user indicated by the user information and the login user according to the user information; and
when the trust value is greater than a trust value threshold, determining a verification result between the user indicated by the user information and the login user is passed, and sending the verification result to the client.

14. The method according to claim 13, wherein the user information at least comprises: user identification information, user reply information and a user question quantity;
acquiring the trust value between the user indicated by the user information and the login user comprises:
querying a user indicated by the user information according to the user identification information; and
acquiring the trust value between the user indicated by the user information and the login user according to the user reply information and the user question quantity.

15. The method according to claim 14, wherein acquiring the trust value between the user indicated by the user information and the login user according to the user reply information and the user question quantity comprises:
according to the user reply information and the user question quantity, acquiring the trust value between the user indicated by the user information and the login user through following formulas:
T=E(Beta(θ|r+1,s+1))r+1/r+s+2
r=r1+r2
wherein, the T is the trust value between the user indicated by the user information and the login user, the r1 is a quantity of questions matched between the user reply information and standard question-answer information, the r2 is a sum of probability matched between the user reply information and the standard question-answer information, the s is a quantity of questions not matched between the user reply information and the standard question-answer information.

16. The method according to claim 13, wherein after determining the verification result between the user indicated by the user information and the login user is passed, the method further comprises:
establishing a following relationship among a target user account, the user indicated by the user information and an anonymous user, a user attribute of the anonymous user is the same as a user attribute of the user indicated by the user information.

17. The method according to claim 12, wherein after acquiring the health information of the concerning user from the user monitoring equipment, the method further comprises:
acquiring an abnormality level of the health abnormality condition when the health information satisfies an abnormal alarm condition; and
outputting alarm information according to an alarm way corresponding to the abnormality level.

18. The method according to claim 17, wherein outputting the alarm information according to the alarm way corresponding to the abnormality level comprises:
sending the alarm information to the login user when the abnormality level is a first abnormality level;
sending the alarm information to a community staff and the login user when the abnormality level is a second abnormality level; and
sending the alarm information to the community staff, first-aid personnel, and the login user when the abnormality level is a third abnormality level.

19. An information interaction method, wherein the method further comprises:
a server acquires health information of a concerning user from user monitoring equipment;
the sever sends the health information to a client of a login user, and the login user is related to the concerning user;
the client displays the concerning user;
the client receives a target input for the concerning user; and
the client displays the health information of the concerning user in response to the target input.

20. (canceled)

21. (canceled)

22. A computing and processing device, wherein the device comprises:
a memory in which a computer-readable code is stored; and
one or more processors, wherein when the computer-readable code is executed by the one or more processors, the computing and processing device executes the information acquisition method according to claim 1.

23. (canceled)

24. (canceled)
